(19) United States
(12) Patent Application Publication
(10) Pub. No.: US 2005/0019348 A1
(43) Pub. Date: Jan. 27, 2005

(54) Marek's disease virus vaccine

(76) Inventors: Sanjay M. Reddy, College Station, TX (US); Blanca M. Lupiani, College Station, TX (US); Richard L. Witter, Okemos, MI (US)

Correspondence Address: USDA-ARS-OFFICE OF TECHNOLOGY TRANSFER;NATIONAL CTR FOR AGRICULTURAL UTILIZATION RESEARCH, 1815 N. UNIVERSITY STREET, PEORIA, IL, 61604, US

(21) Appl. No.: 10/623,891

(22) Filed: Jul. 21, 2003

(51) Int. Cl. C12Q 1/70; C07H 21/04; A61K 39/12; A61K 39/245

(52) U.S. Cl. 424/199.1; 536/23.72; 424/231.1

(57) ABSTRACT

An effective vaccine for Marek's disease may be prepared using a recombinant Marek's disease virus CVI988 transformed with a foreign DNA construct which includes a long terminal repeat sequence of a reticuloendotheliosis virus. This viral agent is effective to elicit an immune response in a chicken to Marek's disease virus without causing a significant degree of pathogenicity in the inoculated bird. Suitable formulations of the vaccine for use in chickens include an effective immunization dosage of this novel viral agent with a pharmaceutically acceptable carrier or diluent.

### BACKGROUND OF THE INVENTION

Field of the Invention

The invention relates to novel vaccines for protecting chickens against infection with Marek's disease virus.

Marek's disease (MD), a highly prevalent and important lymphoproliferative disease of chickens, is controlled in commercial chickens by live virus vaccines consisting of attenuated or naturally avirulent MD-related herpesviruses. Although vaccination programs have been considered highly effective overall, the poultry industry continues to experience losses due to MD. Given the tendency of MD virus to become more virulent with time coupled with the economic pressures confronting the poultry industry, there is still a strong incentive to develop even more efficacious products that will protect better in the face of early challenge with very virulent field strains without causing adverse side effects. This invention relates to a novel vaccine against MD which does in fact provide superior protection and improved safety compared to certain existing commercial vaccines.

There are three distinct serotypes of MD virus found in chickens: (1) serotype 1, the oncogenic form responsible for the disease, including high- and low-virulence MD virus and their attenuated variants; (2) serotype 2, a nononcogenic MD virus; and (3) serotype 3, herpesvirus of turkeys (HVT).

The prototype MD vaccine consists of the serotype 3 virus originally isolated from turkeys as reported in Witter et al. [Am. J. Vet. Res. 31:525-538 (1970)] and Okazaki et al. [U.S. Pat. No. 3,642,574]. Its lack of oncogenicity, self-limiting infection, good replication in vivo and in vitro, availability as cell-free and cell-associated preparations, and high protective efficacy have established HVT as a standard for MD vaccines throughout the world. A commonly used strain of HVT is FC126.

Vaccines produced from the naturally avirulent SB-1 strain [Schat et al., J. Natl. Cancer Inst. 60:1075-1082 (1978) and U.S. Pat. No. 4,160,024], an isolate of a serotype 2 MD virus, have been licensed in the United States since 1984. The SB-1 strain is poorly protective against the highly virulent MDV strains. It is usually used in combination with HVT as a bivalent vaccine since the two viruses together produce greater protection than does either one alone [Schat et al., Avian Pathol. 11:593-606 (1982); Witter, Avian Pathol. 11:49-62 (1982), the contents of which are incorporated by reference herein]. This phenomenon has been termed “protective synergism.” The SB-1+HVT bivalent vaccine represents about 18% of the United States market for MD vaccines at present and is considered to be among the most efficacious of the various MD products available. However, sporadic losses occur despite its use.

Another MD vaccine produced from strain CVI988clone C (CVI988/C) has been licensed for commercial use in the United States. This vaccine was derived from a mildly virulent serotype 1 MD virus attenuated by serial passage in tissue culture and has been reported by De Boer et al. [Avian Dis. 30:276-283 (1986)]. A further passaged derivative of CVI988/C, identified as CVI988/C/R6, has also been described by De Boer et al. [Advances in Marek's Disease Research, pp. 405-413 (1988)]. More recently, the original low-passage strain, designated CV1988/Rispens, which has been in commercial use in other countries for a number of years, was found to be highly effective against challenge with several very virulent MD virus strains by Witter et al. [4th Int'l. Symp. Marek's Disease, pp. 315-319 (1992)].

An experimental vaccine derived from Md11, a very virulent serotype 1MD field isolate, was reported by Witter, supra. Md11 was attenuated by 75 serial passages in cell culture, and the resultant vaccine designated Md11/75C. This vaccine has been shown to provide good protection against challenge with Md5 and most other highly virulent MD viruses tested; but it was less efficacious against challenge with the JM/102W strain, a prototype MD virus effectively protected against by HVT and SB-1 vaccines. Furthermore, its efficacy was consistently lower in chicks with HVT antibody.

In U.S. Pat. No. 4,895,717, Witter disclosed a revertant derivative of Md11/75C which was referred to as Md11/75C/R2. Md11/75C/R2 was shown to be superior to several other monovalent vaccines and was the equal of a bivalent (HVT+SB-1) vaccine [Witter, Avian Dis. 31:752-765 (1987)]. However, the inherent pathogenicity of serotype 1 viruses and the potential of attenuated strains to revert to greater pathogenicity [Witter et al., Avian Pathol. 13:75-92 (1984)] are factors to be considered in the licensing of such products. A clone derived from further passages of the Md11/75C/R2 strain, designated Md11/75C/R2/23 (or R2/23), was found by Witter et al. [Avian Dis., 35:877-891 (1991)] to possess the highly protective nature of the parent strain without its residual pathogenicity.

Witter also described another MD vaccine derived from 301B/1, a nonpathogenic serotype 2 field isolate, in U.S. Pat. No. 4,895,718, the contents of which are incorporated by reference herein. Strain 301B/1 possessed superior replicative ability to SB-1, as well as greater protectivity against challenge to viruses.

Still other concerns have arisen over the use of some MD vaccines. As indicated, bivalent vaccines composed of MD virus serotypes 2 and 3 are currently widely used in the United States and have provided excellent protection against very virulent MD strains. However, use of such vaccines containing serotype 2 MD virus may lead to increased mortality from another disease, lymphoid leukosis. This enhancement of lymphoid leukosis in avian leukosis virus infected chickens resulting from vaccination with products containing serotype 2 MD virus has been an unfortunate deterrent to their expanded use.

A recombinant Marek's disease virus, referred to as RM1, having the long terminal repeats of reticuloendotheliosis virus stably integrated into the repeat short (RS) regions of its genome was also described. This strain was generated at the USDA-ARS-ADOL from a pathogenic serotype 1 Marek's disease virus strain JM [Witter et al., 1997, Avian Dis., 41:407-421, and Jones et al., 1996, J. Virology, 70(4):2460-2467]. However, while the RM1 strain has been shown to provide a level of protection similar or superior to that of CVI988, it has also been associated with residual pathogenicity, causing thymic atrophy in treated birds.

Thus, although HVT, SB-1, CVI988, CVI988/C, Md11/75C, Md11/75C/R2 and 301B/1 are all effective against certain MD viruses, none of these vaccines protect optimally against all MD challenge viruses in all chickens. Moreover, these vaccines have exhibited reduced efficacy against some of the more recently isolated very virulent strains of MD virus. To avert any large-scale outbreaks of MD in the future, the need exists to develop improved vaccines effective against the very virulent strains of MD virus.

### SUMMARY OF THE INVENTION

We have now discovered that an effective vaccine for Marek's disease may be prepared using a recombinant Marek's disease virus strain CVI988 transformed with a foreign DNA construct which includes a long terminal repeat sequence of a reticuloendotheliosis virus. This viral agent is effective to elicit an immune response in a chicken to Marek's disease virus without causing a significant degree of pathogenicity in the inoculated bird. Suitable formulations of the vaccine for use in chickens include an effective immunization dosage of this novel viral agent with a pharmaceutically acceptable carrier or diluent.

In accordance with this discovery, it is an object of the invention to provide a novel, highly protective vaccine against MD in chickens.

It is also an object of the invention to provide a vaccine which provides greater protection against very virulent strains of Marek's disease virus than those vaccines presently in commercial use.

It is another object of the invention to improve the viability and productivity of chickens, particularly broilers and layers, and to reduce economic losses in the poultry industry caused by Marek's disease.

Other objects and advantages of this invention will become readily apparent from the ensuing description.

### BRIEF DESCRIPTION OF THE DRAWINGS

FIG. 1 shows a schematic organization of MDV genome. (A) The genome of MDV consists of unique long (UL) region flanked by inverted repeats, terminal repeat long (TRL), internal repeat long (IRL), and a unique short region (US) also flanked by two inverted repeats, internal repeat short (IRS and terminal repeat short (TRS). (B) Schematic representation of the overlapping cosmid clones generated to rescue an infectious virus from a very virulent strain of MDV. The restriction sites used to generate the cosmid clones and their positions are indicated. Transfection of SN5, P89, SN16, A6 and B40 into cells in culture resulted in the generation of infectious MDV.

FIG. 2 shows the generation of the B40-Pac cosmid used to generate CVRM vaccine. The B40 cosmid represents the right-most fragment of Md5 genome. The Pac I fragment of RM1 was used to replace the corresponding region in the B40 cosmid to generate B40-Pac. The resulting cosmid was packaged using Gigapack III Gold Packaging Extracts (Stratagene) using standard protocols. The recombinant cosmid, B40-Pac was screened for the presence of two copies of LTR (long terminal repeats). The positive clones were propagated in HB101 bacteria and digested with Not I restriction enzyme and co-transfected into cells in culture along with nucleocapsid DNA isolated from CVI988 strain (Intervet, Delaware). The DNA from CVI988 recombined with the B40-Pac by homologous recombination to generate CVRM vaccine. The CVRM strain, due to the presence of LTR insert, grew at a faster rate than parental vaccine CVI988 thus out competing the parental strain.

### DEPOSIT OF BIOLOGICAL MATERIAL

Recombinant Marek's disease virus clone CVRM-2 has been deposited under the provisions of the Budapest Treaty in the American Type Culture Collection (ATCC), 10801 University Blvd., Manassass, Va., 20110-2209, USA, on Jan. 22, 2003, and has been assigned Accession No. ATCC PTA-4945.

### Definitions

The following terms are employed herein:

Cloning. The selection and propagation of (a) genetic material from a single individual, (b) a vector containing one gene or gene fragment, or (c) a single organism containing one such gene or gene fragment.

Cloning Vector. A plasmid, virus, retrovirus, bacteriophage, cosmid, artificial chromosome (bacterial or yeast), or nucleic acid sequence which is able to replicate in a host cell, characterized by one or a small number of restriction endonuclease recognition sites at which the sequence may be cut in a predetermined fashion, and which may contain an optional marker suitable for use in the identification of transformed cells, e.g., tetracycline resistance or ampicillin resistance. A cloning vector may or may not possess the features necessary for it to operate as an expression vector.

Codon. A DNA sequence of three nucleotides (a triplet) which codes (through mRNA) for an amino acid, a translational start signal, or a translational termination signal. For example, the nucleotide triplets TTA, TTG, CTT, CTC, CTA, and CTG encode for the amino acid leucine, while TAG, TAA, and TGA are translational stop signals, and ATG is a translational start signal.

DNA Coding Sequence. A DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences and cDNA from eukaryotic mRNA. A polyadenylation signal and transcription termination sequence will usually be located 3′ to the coding sequence.

DNA Construct. Artificially constructed (i.e., non-naturally occurring) DNA molecules useful for introducing DNA into host cells, including chimeric genes, expression cassettes, and vectors.

DNA Sequence. A linear series of nucleotides connected one to the other by phosphodiester bonds between the 3′ and 5′ carbons of adjacent pentoses.

Expression. The process undergone by a structural gene to produce a polypeptide. Expression requires transcription of DNA, post-transcriptional modification of the initial RNA transcript, and translation of RNA.

Expression Cassette. A nucleic acid sequence within a vector which is to be transcribed, and a promoter to direct the transcription. The expression cassette may contain one or more unrelated DNA sequences encoding one or more peptides of interest.

Expression Control Sequence. Expression control sequences are DNA sequences involved in any way in the control of transcription or translation and must include a promoter. Suitable expression control sequences and methods of making and using them are well known in the art.

Expression Vector. A replicon such as a plasmid, virus, retrovirus, bacteriophage, cosmid, artificial chromosome (bacterial or yeast), or nucleic acid sequence which is able to replicate in a host cell, characterized by a restriction endonuclease recognition site at which the sequence may be cut in a predetermined fashion for the insertion of a heterologous DNA sequence. An expression vector has a promoter positioned upstream of the site at which the sequence is cut for the insertion of the heterologous DNA sequence, the recognition site being selected so that the promoter will be operatively associated with the heterologous DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Fusion Protein. A protein produced when two heterologous genes or fragments thereof coding for two different proteins not found fused together in nature are fused together in an expression vector. For the fusion protein to correspond to the separate proteins, the separate DNA sequences must be fused together in correct translational reading frame.

Gene. A segment of DNA which encodes a specific protein or polypeptide, or RNA.

Genome. The entire DNA of an organism. It includes, among other things, the structural genes encoding for the polypeptides of the substance, as well as operator, promoter and ribosome binding and interaction sequences.

Heterologous DNA. A DNA sequence inserted within or connected to another DNA sequence which codes for polypeptides not coded for in nature by the DNA sequence to which it is joined. Allelic variations or naturally occurring mutational events do not give rise to a heterologous DNA sequence as defined herein.

Hybridization. The pairing together or annealing of single stranded regions of nucleic acids to form double-stranded molecules.

Mutant. As used herein, “mutant” refers to any stable virus whose functional properties are different from the parent strain.

Nucleotide. A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. The base characterizes the nucleotide. The four DNA bases are adenine (“A”), guanine (“G”), cytosine (“C”), and thymine (“T”). The four RNA bases are A, G, C, and uracil (“U”).

Operably Encodes or Associated. Operably encodes or operably associated each refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Phage or Bacteriophage. Bacterial virus many of which include DNA sequences encapsidated in a protein envelope or coat (“capsid”). In a unicellular organism a phage may be introduced by a process called transfection.

Plasmid. A non-chromosomal double-stranded DNA sequence comprising an intact “replicon” such that the plasmid is replicated in a host cell. When the plasmid is placed within a unicellular organism, the characteristics of that organism may be changed or transformed as a result of the DNA of the plasmid. A cell transformed by a plasmid is called a “transformant.”

Polypeptide. A linear series of amino acids connected one to the other by peptide bonds between the alpha-amino and carboxy groups of adjacent amino acids.

Promoter. A DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible.

Reading Frame. The grouping of codons during translation of mRNA into amino acid sequences. During translation the proper reading frame must be maintained. For example, the DNA sequence may be translated via mRNA into three reading frames, each of which affords a different amino acid sequence.

Recombinant DNA Molecule. A hybrid DNA sequence comprising at least two DNA sequences, the first sequence not normally being found together in nature with the second.

Ribosomal Binding Site. A nucleotide sequence of mRNA, coded for by a DNA sequence, to which ribosomes bind so that translation may be initiated. A ribosomal binding site is required for efficient translation to occur. The DNA sequence coding for a ribosomal binding site is positioned on a larger DNA sequence downstream of a promoter and upstream from a translational start sequence.

Replicon. Any genetic element (e.g., plasmid, chromosome, virus) that functions as an autonomous unit of DNA replication in vivo, i.e., capable of replication under its own control.

Start Codon. Also called the initiation codon, is the first mRNA triplet to be translated during protein or peptide synthesis and immediately precedes the structural gene being translated. The start codon is usually AUG, but may sometimes also be GUG.

Structural Gene. A DNA sequence which encodes through its template or messenger RNA (mRNA) a sequence of amino acids characteristic of a specific polypeptide.

Transform or transfect. To change in a heritable manner the characteristics of a host cell in response to DNA foreign to that cell. An exogenous DNA has been introduced inside the cell wall or protoplast. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eucaryotic cells, a stably transformed cell is one in which the exogenous DNA has been integrated into a chromosome so that it is inherited by daughter cells through chromosome replication. This stability is demonstrated by the ability of the eucaryotic cell to establish cell lines or clones comprised of a population of daughter cells containing the exogenous DNA.

Transcription. The process of producing mRNA from a structural gene.

Translation. The process of producing a polypeptide from mRNA.

Vaccine. A vaccine is defined herein in its broad sense to refer to any type of biological agent in an administratable form capable of stimulating an immune response in an animal inoculated with the vaccine. For purposes of this invention, the vaccine may comprise either the virus itself or an immunogenic (antigenic) component of the virus. The vaccine may also be produced from a vector having inserted therein a gene which encodes an immunogenic component of the virus.

### DETAILED DESCRIPTION OF THE INVENTION

The present invention provides Recombinant Marek's disease virus which has a substantially isolated long terminal repeat derived from the genome of a reticuloendotheliosis virus, incorporated into its genome. These recombinants are effective to elicit an immune response in a chicken to Marek's disease virus without causing a significant degree of pathogenicity in the chicken. As used herein “without causing a significant degree of pathogenicity” in the chicken is defined as no gross MD specific lesions being observable with the naked eye in the inoculated chicken, even in highly susceptible chicken lines.

The recombinant Marek's disease virus of this invention is produced by transformation of Marek's disease virus serotype 1 strain CVI988 or any of its clones or serially passaged strains, which are collectively referred to herein as strains CVI988/X. Thus, as used herein CVI988/X includes but is not limited to the previously described original low-passage strain, CVI988/Rispens (Rispens et al., 1972, Avian Dis., 16:106-125 and 126-138), strain CVI988 clone C (CVI988/C) (De Boer, U.S. Pat. No. 4,673,572, and De Boer et al., 1986, Avian Dis. 30:276-283), and CVI988/C/R6 (De Boer et al., 1988, Advances in Marek's Disease Research, pp. 405-413). The contents of each of the above-mentioned publications/patents are incorporated by reference herein. In accordance with the invention, this Marek's disease virus strain CVI988/X is recombined with a foreign DNA construct which includes a long terminal repeat (LTR) sequence of a reticuloendotheliosis virus (REV).

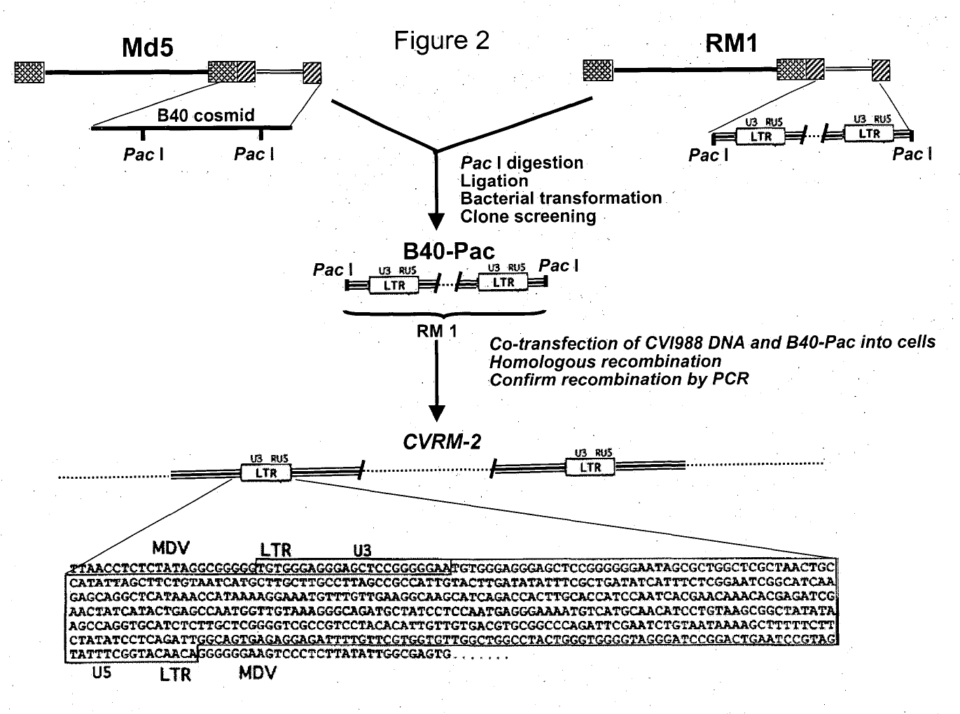

Recombination of the Marek's disease virus strain CVI988 was originally effected using Marek's disease virus serotype 1 strain RM1 as a source of the reticuloendotheliosis virus LTRs. Strain RM1 is a recombinant Marek's disease virus which has the LTRs of reticuloendotheliosis virus integrated into its genome. As shown in FIG. 2 and described in detail in Example 1, the reticuloendotheliosis viral LTR was excised from the purified RM1 viral DNA by Pac I digestion and inserted into a shuttle vector, B40, prepared from a very virulent strain of Marek's disease virus, Md5. The resultant recombinant vector, B40-Pac, was used for insertion of the LTRs into the Marek's disease virus strain CVI988. To generate recombinant Marek's disease virus with the LTRs, the purified viral DNA of Marek's disease virus strain CVI988 was co-transfected into chicken or duck embryonic fibroblast (CEF or DEF) cells with Not I digested recombinant vector. Recombinant viruses having the LTRs integrated into their genome replicated faster than the parental CVI988 strain and were recovered and isolated. Without being bound by theory, it is believed that this increased rate of replication is the result of the insertion of the reticuloendotheliosis virus LTR into the genome of the Marek's disease virus upstream of the ICP4 gene.

In an alternative embodiment, the Marek's disease virus CVI988/X may be transformed with isolated reticuloendotheliosis virus LTRs from sources other than the above-mentioned RM1 strain. For instance, the insertion site of the LTR in the RM1 strain of Marek's disease virus has been shown to be between IRL and IRS of the genome (Jones et al., 1996, Retroviral insertional activation in a herpesvirus: transcriptional activation of US genes by an integrated long terminal repeat in a Marek's disease virus clone, J. Virology, 70(4):2460-2467). This corresponds approximately to position 152,745 of the Md5 strains of Marek's disease virus. This region is located within a 1,704 base pair long EcoRI fragment (nucleotides 152, 198-153, 902) of serotype 1 Md5 (Tulman et al., 2000, The genome of a very virulent Marek's disease virus, J. Virology, 74(17):7980-7988). This 1,704 bp EcoRI fragment can be cloned into a plasmid vector lacking DraIII restriction endonuclease site and used as a transfer vector for introduction of any LTR in to the MDV genome. This EcoRI fragment has a unique DraIII restriction site located 10 bp upstream of the LTR location in RM1. The LTRs can be inserted into the DraIII site of the 1,704 base pair EcoRI fragment to generate the LTR transfer vector. In order to generate recombinant MDV with LTR insertions, the transfer vector should be linearized with EcoRI, extracted with phenol and chloroform and precipitated with ethanol. Co-transfection of the linearized transfer vector along with DNA-from any serotype 1 MDV strain into permissible cells in culture, will result in the introduction of LTR sequences into the MDV genome by homologous recombination. The resulting recombinant virus, MDV with LTR, generally grow more rapidly than parental MDV strain and thus there is no need for plaque purification. However, if there is no growth advantage, the recombinant viruses have to be plaque purified and screened for the presence of LTR sequences using PCR.

In another alternative embodiment, recombinant Marek's disease virus having the reticuloendotheliosis virus LTRs may be prepared from any Marek's disease virus, including other CVI988/X strains, using the deposited CVRM-2. In brief, the purified DNA of the CVRM may be digested with Pac I, and the Pac I fragment containing the reticuloendotheliosis virus LTR is inserted into any vector containing Pac I sites. This resultant vector may then be co-transfected with any serotype 1 Marek's disease virus, including another CVI988/X strain, and recombinant virus recovered using the same procedures described above.

A variety of reticuloendotheliosis viral LTRs are suitable for use herein. Numerous suitable reticuloendotheliosis viral LTRs have been isolated and described, and include but are not limited to those described by Kost et al. (1993, Retrovirus insertion into herpesvirus: characterization of a Marek's disease virus harboring a solo LTR, Virology, 192:161-169), Ridgway (1992, Reticuloendotheliosis virus long terminal repeat elements are efficient promoters in cells of various species and tissue origin, including human lymphoid cells, Gene, 121:213-218), Boerkoel and Kung [1992, Transcriptional interaction between retroviral long terminal repeats (LTRs): mechanism of 5′ LTR suppression and 3′ LTR promoter activation of c-myc in avian B-cell lymphomas, J. Virol., 66:4814-4823], Hippenmeyer and Krivi (1991, Gene expression from heterologous promoters in a replication-defective avian retrovirus vector in quail cells, Poult. Sci., 70:982-92); Ridgway et al. (1989, Transient expression analysis of the reticuloendotheliosis virus long terminal repeat element, Nucleic Acids Res., 17:3199-3215), Embretson and Temin (1987, Transcription from a spleen necrosis virus 5′ long terminal repeat is suppressed in mouse cells, J. Virol., 61:3454-3462), Notani and Sauerbier (1987, Sequence instability in the long terminal repeats of avian spleen necrosis virus and reticuloendotheliosis virus, J. Mol. Evol., 25:241-247), Robinson and Gagnon (1986, Patterns of proviral insertion and deletion in avian leukosis virus-induced lymphomas, J. Virol., 57:28-36), and Ridgway et al., (1985, In vitro transcription analysis of the viral promoter involved in c-myc activation in chicken B lymphomas: detection and mapping of two RNA initiation sites within the reticuloendotheliosis virus long terminal repeat, J. Virol., 54:161-170). The contents of each of the publications referred to above are incorporated by reference herein. In addition, numerous LTR sequences are available in GenBank and other genomic databases and can be synthesized by PCR using LTR specific primers. The PCR amplified sequences can then be inserted in any of the two transfer vectors described as indicated above.

The reticuloendotheliosis viral LTR nucleic acid sequences disclosed herein, or their biologically functional equivalents, can be used in accordance with the present invention. The phrase “biologically functional equivalents” as used herein, denotes nucleic acid sequences exhibiting the same or similar biological activity/immunoprotective activity as the above-mentioned reticuloendotheliosis viral LTR nucleic acid sequences (i.e., when introduced into the CVI988 Marek's disease virus host in a functionally operable manner they elicit a protective immune response without causing a significant degree of pathogenicity in the chicken).

For example, the nucleic acid sequences described herein can be altered by base substitutions, insertions, additions, or deletions to produce biologically functionally equivalent nucleic acids that retain promoter or enhancer activity.

The variants of the genomic DNAs or cDNAs (if obtained by RT-PCR from RNA), contemplated herein should possess more than 75% homology, preferably more than 85% homology, and most preferably more than 95% homology, to the naturally occurring reticuloendotheliosis viral LTRs discussed herein.

The vaccine of the recombinant Marek's disease virus of the invention may be prepared as a cell-free preparation, or in the preferred embodiment, as a cell-associated preparation. A cell-associated vaccine can be prepared directly from in vitro culture of the live viral agents in a suitable growth medium, such as chicken embryo fibroblasts as described by Witter [U.S. Pat. No. 4,895,718, the contents of which are incorporated by reference herein). Alternatively, to prepare cell-free virus inocula, cells from infected host tissue or cell culture are sonicated or otherwise disrupted as previously described. The cellular debris is removed by centrifugation and the centrifugate recovered as the inoculum. Moreover, while the preferred vaccine is a viable virus, it is also envisioned that the vaccine may be prepared from the killed virus or from immunogenic components separated from the virus, although such processing would incur significantly greater costs. For example, a subunit vaccine can be prepared by separating from the killed virus one or more purified viral proteins identified as having immunogenic properties.

The viral agent is prepared for administration by formulation in an effective immunization dosage with a pharmaceutically acceptable carrier or diluent, such as physiological saline or tissue culture medium. The expression “effective immunization dosage” is defined as being that amount which will induce immunity in a chicken against challenge by a virulent strain of Marek's disease virus. Immunity is considered as having been induced in a population of chickens when the level of protection for the population is significantly higher than that of an unvaccinated control group (measured at a confidence level of at least 80%, preferably measured at a confidence level of 95%). One measure of the level of protection is the protective index (PI), which is calculated as the incidence of Marek's disease in unvaccinated, Marek's disease virus challenged controls minus the incidence of Marek's disease in vaccinated, Marek's disease virus challenged groups, and the difference divided by the percent of Marek's disease in unvaccinated, Marek's disease virus challenged controls, with the result multiplied by 100. Typically, the vaccine will contain at least about 200 PFU (plaque-forming units) of the virus, and preferably between about 2000 and 5000 PFU. The vaccine can be effectively administered anytime after the chicken attains immunocompetence, which is at about the 18th day of incubation (3 days prehatch); but it is normally administered by inoculation within 24-48 hours after hatching. Alternatively, the recombinant viral DNA may be administered as a DNA vaccine as described by Tischer et al. (2002, J. Gen. Virology, 83:2367-2376, the contents of which are incorporated by reference herein).

Appropriate adjuvants as known in the art may also be included in the vaccine formulation. In many cases, the vaccinal efficacy can be enhanced by combining the recombinant Marek's disease viruses of the invention with other viral agents into bivalent or polyvalent vaccines.

The following examples are intended only to further illustrate the invention and are not intended to limit the scope of the invention which is defined by the claims.

### EXAMPLE 1

Generation of CVRM-2 Recombinant Virus

A recombinant serotype 1 Marek's disease virus, strain CVRM-2, was generated by insertion of the long terminal repeat sequence of a reticuloendotheliosis virus into the genome of the CVI988 strain of Marek's disease virus.

The LTR was inserted using Marek's disease virus serotype 1 strain RM1. Strain RM1 is a recombinant Marek's disease virus having the long terminal repeats of reticuloendotheliosis virus stably integrated into the repeat short (RS) regions of its genome. To generate a vaccine that provides improved protection over strain CVI988 but which does not exhibit the residual pathogenicity of RM1, a chimeric virus between CVI988 and RM1 was generated. This new vaccine, called CVRM, contains most of the CVI988 genome and has the same LTR insertion present in RM1.

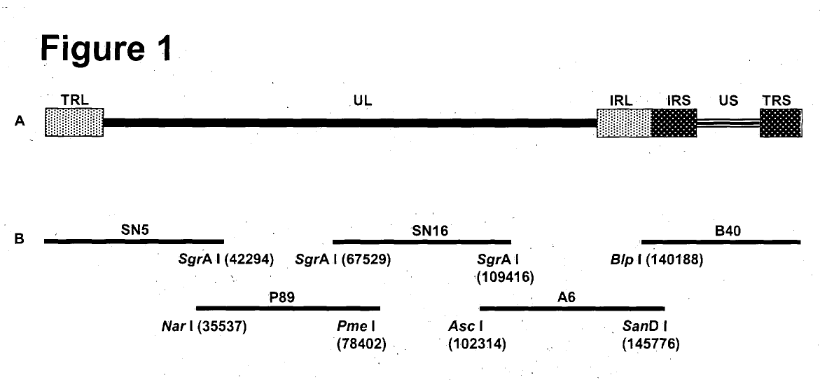

To develop the CVRM vaccine virus, a previously developed cosmid clone (B40) of a very virulent strain of Marek's disease virus, Md5, (Reddy et al., 2002, PNAS USA, 99:7054-7059) was used as a shuttle vector (FIG. 1). Briefly, as shown in FIG. 1, to clone the B40 fragment, Md5 viral DNA was digested with Blp I (position 140189), blunt ended with T4 DNA polymerase and ligated to Not I linkers. Ligated DNA was digested with Not I and inserted into Not I digested SuperCos I cosmid vector (Stratagene, La Jolla, Calif.). Ligation reactions were packaged using the Gigapack® III Gold Packaging Extract (Stratagene, La Jolla, Calif.) and inserted into HB101 E. coli (Life Technologies Inc., Rockville, Md.). To identify bacterial clones containing correct viral DNA inserts, plasmid DNA was isolated, subjected to restriction enzyme digestion using different restriction enzymes and the patterns obtained compared to those estimated from the Md5 sequence (Tulman et al., 2000, J. Virol., 74:7980-7988). Clones with the correct restriction pattern were selected for further use.

As shown in FIG. 2, the MDV serotype 1 genome contains two Pac I sites (positions 151493 and 166952) in the RS region of the genome and none in the unique short (US) region. The retrovirus LTR insertion in RM1 is present within this two Pac I sites. In order to generate a B40 vector with the RM1 LTR insertions, B40 was digested with Pac I and the DNA fragment excised replaced by the corresponding Pac I fragment of RM1. The resulting recombinant B40 vector, B40-Pac, was screened by PCR using primers specific for the retroviral LTR, and a cosmid with two copies of the LTR. (one in each RS region) selected for further use.

To generate the CVRM virus, purified viral DNA of the MDV serotype 1 vaccine strain CVI988 (commercial strain purchased from Intervet, Delaware) was co-transfected into CEF or DEF cells together with Not I digested cosmid B40-Pac as previously described (Reddy et al., 2002, ibid). Recombinant CVI988 viruses containing the LTR insertion from RM1 replicated faster than parental CVI988 strain virus and soon became the predominant viral population. In addition, recombinant viruses were released into the supernatant as cell free virus at a higher rate than parental CVI988 strain. Two recombinant strains, CVRM-1 and CVRM-2, were recovered and further propagated. CVRM-1 virus stocks were made by passing the infected cells in cultured cells for 7 passages, while CVRM-2 virus stocks were made by passing only supernatants of infected cells into subsequent passages at passage 2 and 3 and subsequently by passing infected cells. The final stocks of CVRM-2 were made at passage 8.

A partial map of the CVRM genome showing the location of the insertion of the LTR therein is shown in Sequence ID No. 1, while the sequence of the reticuloendotheliosis virus LTR per se is shown in Sequence ID No. 2.

Efficacy.

Protection studies were conducted in the MD susceptible F1 progeny (15×7) of Avian Disease and Oncology Laboratory line 15I₅ males and line 7₁ females. The chickens used in the experiments were derived from breeder hens that were vaccinated with all three serotypes of MDV (Ab-positive). These chickens were randomly sorted into groups and held in modified Horsfall-Bauer isolators for 8 weeks. The vaccines were administered at day of age and were challenged with a very virulent plus strain (vv+), 648A (Witter, 1997, Avian Dis. 41:149-163) at day 6 of age (5 days post vaccination). All chickens that died during the trial or were killed at the end of experiment were examined for typical gross MD lesions. The results are summarized in Table 1.

The contents of each of the publications referred to hereinabove are incorporated by reference herein.

Recombinant Marek's disease virus clone CVRM-2 has been deposited under the provisions of the Budapest Treaty in the American Type Culture Collection (ATCC), 10801 University Blvd., Manassass, Va., 20110-2209, USA, on Jan. 22, 2003, and has been assigned Accession No. ATCC PTA-4945.

It is understood that the foregoing detailed description is given merely by way of illustration and that modifications and variations may be made therein without departing from the spirit and scope of the invention.

TABLE 1
Protection Studies of CVRM vaccines

| | Experiment 1 | | Experiment 2 | | Experiment 3 | | |
|---|---|---|---|---|---|---|---|
| Virus/Challenge | MD | PI | MD | PI | MD | PI | Average PI |
| control | 0/17 | NA | 0/13 | NA | 0/3 | NA | |
| CVRM1 | 0/10 | NA | 0/9 0/8 | NA | ND | NA | |
| CVRM2 | 0/10 | NA | 0/9 0/8 | NA | 0/10 | NA | |
| CVI988 | 0/10 | NA | ND | NA | 0/5 | NA | |
| CVRM1/648A | 1/17 | 94.1 | 4/17 | 76.5 | ND | ND | 85.3 |
| CVRM2/648A | 4/17 | 76.5 | 3/17 | 82.4 | 3/17 | 82.4 | 79.5 |
| CVI988/648A | 7/17 | 58.8 | 6/17 | 64.7 | 3/10 | 70 | 64.5 |
| None/648A | 15/17 | NA | 17/17 | NA | 10/10 | NA | |

Chicken: 15 × 7
Antibody status: maternal antibody positive
Vaccination: day 1
Challenge: day 6
Termination: 8 weeks
PI (protection index): indicates percentage of chickens protected form MD

1. A viral agent comprising a recombinant Marek's disease virus CVI988/X stably transformed with a foreign DNA construct which comprises a long terminal repeat sequence of a reticuloendotheliosis virus, wherein said viral agent is effective to elicit an immune response in a chicken to Marek's disease virus without causing a significant degree of pathogenicity in said chicken, and further wherein said lone terminal repeat sequence is inserted upstream of the ICP4 gene of said Marek's disease virus.

2. The viral agent of claim 1 wherein said long terminal repeat sequence comprises Sequence ID No. 2.

3. The viral agent of claim 1 wherein said long terminal repeat sequence comprises a Pac I excised DNA segment from a Marek's disease virus having all of the identifying characteristics of strain ATCC PTA-4945.

4. (canceled).

5. The viral agent of claim 1 comprising a Marek's disease virus having all of the identifying characteristics of strain ATCC PTA-4945.

6. The viral agent of claim 1 wherein said recombinant Marek's disease virus is viable.

7. The viral agent of claim 1 wherein said Marek's disease virus is cell associated.

8. A vaccine comprising the viral agent of claim 1 in an amount effective to elicit a protective immune response in a chicken to Marek's disease virus and a pharmaceutically acceptable carrier or diluent.

9. A method for protecting a chicken against Marek's disease comprising inoculating said chicken with a vaccine comprising a viral agent in an amount effective to elicit a protective immune response in a chicken to Marek's disease virus and a pharmaceutically acceptable carrier or diluent, wherein said viral agent comprises a recombinant Marek's disease virus CVI988/X transformed with a foreign DNA construct which comprises a long terminal repeat sequence of a reticuloendotheliosis virus and said long terminal repeat is inserted upstream of the ICP4 gene of said Marek's disease virus, and further wherein said viral agent is effective to elicit a protective immune response in a chicken to Marek's disease virus without causing a significant degree of pathogenicity in said chicken.

10. The method of claim 9 wherein said long terminal repeat sequence comprises a Pac I excised DNA segment from a Marek's disease virus having all of the identifying characteristics of strain ATCC PTA-4945.

11. (canceled).

12. The method of claim 9 wherein said viral agent comprises a Marek's disease virus having all of the identifying characteristics of strain ATCC PTA-4945.

13. The method of claim 9 wherein said viral agent is viable.

14. The method of claim 9 wherein said vaccine is cell associated.

15. A method of making a viral agent effective for protecting a chicken against Marek's disease comprising transforming a Marek's disease virus strain CVI988 with a foreign DNA construct which comprises a long terminal repeat sequence of a reticuloendotheliosis virus, and further wherein said long terminal repeat is inserted upstream of the ICP4 gene of said Marek's disease virus.


SEQUENCE LISTING (2 sequences)

SEQ ID NO: 1 - DNA, 585 bp, recombinant MDV Strain RM1
ttaacctctctataggcgggggtgtgggagggagctccgggggaatgtgggagggagctccggggggaatagcgctggctcgctaactgccatattagcttctgtaatcatgcttgcttgccttagccgccattgtacttgatatatttcgctgatatcatttctcggaatcggcatcaagagcaggctcataaaccataaaaggaaatgtttgttgaaggcaagcatcagaccacttgcaccatccaatcacgaacaaacacgagatcgaactatcatactgagccaatggttgtaaagggcagatgctatcctccaatgagggaaaatgtcatgcaacatcctgtaagcggctatataagccaggtgcatctcttgctcggggtcgccgtcctacacattgttgtgacgtgcggcccagattcgaatctgtaataaaagctttttcttctatatcctcagattggcagtgagaggagattttgttcgtggtgttggctggcctactgggtggggtagggatccggactgaatccgtagtatttcggtacaacaggggggaagtccctcttatattggcgagtg

SEQ ID NO: 2 - DNA, 533 bp, Reticuloendothelial virus
  repeat_region (1)..(533): Long Terminal Repeat from Reticuloendothelial Virus
tgtgggagggagctccgggggaatgtgggagggagctccggggggaatagcgctggctcgctaactgccatattagcttctgtaatcatgcttgcttgccttagccgccattgtacttgatatatttcgctgatatcatttctcggaatcggcatcaagagcaggctcataaaccataaaaggaaatgtttgttgaaggcaagcatcagaccacttgcaccatccaatcacgaacaaacacgagatcgaactatcatactgagccaatggttgtaaagggcagatgctatcctccaatgagggaaaatgtcatgcaacatcctgtaagcggctatataagccaggtgcatctcttgctcggggtcgccgtcctacacattgttgtgacgtgcggcccagattcgaatctgtaataaaagctttttcttctatatcctcagattggcagtgagaggagattttgttcgtggtgttggctggcctactgggtggggtagggatccggactgaatccgtagtatttcggtacaaca